(19) United States
(12) Patent Application Publication
(10) Pub. No.: US 2015/0367106 A1
(43) Pub. Date: Dec. 24, 2015

(54) BODY PASSAGE DEVICE

(71) Applicant: JET PREP LTD., Yoqneam (IL)

(72) Inventors: David Nitsan, Tel-aviv (IL); Nadav Ben-Da'at, Karkur (IL)

(21) Appl. No.: 14/766,081

(22) PCT Filed: Feb. 6, 2014
(86) PCT No.: PCT/IB2014/000722

§ 371 Date: Aug. 5, 2015

Related U.S. Application Data
(60) Provisional application No. 61/761,846, filed on Feb. 7, 2013.

(51) Int. Cl. A61M 25/09; A61M 3/02

(52) U.S. Cl. CPC A61M 25/0905; A61M 3/0279; A61M 2025/09183; A61B 1/126

(57) ABSTRACT

A body passage device and method for irrigating and/or cleaning the working channel of an endoscope or patient tissue is provided. The body passage device and method limits unwanted lateral and/or vertical movement of the spray head, guidewire, and/or tube surrounding the guidewire. Body passage devices and methods are provided that allow the guidewire and/or spray head to be extended to universally adapt to a variety of different length endoscope working channels.

This application claims priority to U.S. Provisional Application Ser. No. 61/761,846 filed Feb. 7, 2013. This application is incorporated herein by reference into the present disclosure.

### TECHNICAL FIELD

The present disclosure generally relates to a device for irrigating, aspirating, or cleaning patient tissue and/or the working channel or conduit of an endoscope.

### BACKGROUND

Endoscopes play a critical role in medical diagnosis and treatment. Often, endoscopes can be used to illuminate, examine and document difficult-to-access areas and other body regions to facilitate diagnosis and treatment of hidden diseases. Endoscopes can also assist in enhancing the planning and preparation of invasive operations.

Many endoscopes contain a working channel or conduit that allows the clinician to insert elongated medical devices, such as body passage cleaning devices, through the working channel and into a body passage (e.g., colon, esophagus, etc.). These elongated medical devices can be used to irrigate, clean, biopsy, repair or remove patient tissue. Some endoscopes have working channels that allow insertion of elongated medical devices for irrigation, aspiration and/or cleaning of patient tissue or for the removal of debris from the working channel of the

Some elongated medical devices have spray heads on their distal ends that make irrigation and/or cleaning of the working channel or patient tissue easier. These spray heads are often disposed on a guidewire that allows the clinician to move the spray head in and out of the working channel of the endoscope as well as beyond the endoscope to the patient tissue or body cavity (e.g., colon, esophagus, etc.).

Unfortunately, there can be unwanted lateral or vertical movement of the guide wire and/or the spray head relative to the working channel and any tube surrounding the guidewire. This unwanted movement can reduce the precision in irrigating or cleaning of the working or patient tissue. There can also be less precision when positioning the elongated medical device relative to other medical devices that can also be disposed in the working channel of the endoscope.

Some working channels of the endoscopes have varying length, even those endoscopes that are the same make and model. Therefore, adjustment of the elongated medical device for irrigation, aspiration and/or cleaning of the working channel or patient tissue may become difficult. Making body passage devices for each different type of endoscope with the varied length in the working channel can be costly.

Therefore, there is a need for a body passage device and method for irrigating and/or cleaning the working channel of an endoscope or patient tissue that limits unwanted lateral or vertical movement of the spray head, guidewire, and/or tube surrounding the guidewire. Body passage devices that allow the guidewire and/or spray head to be extended to universally adapt it to a variety of different length endoscope working channels or lumens would also be beneficial.

### SUMMARY

New body passage devices and methods are provided for irrigating and/or cleaning the working channel of an endoscope or patient tissue that allows precision irrigation and/or cleaning by limiting unwanted lateral and/or vertical movement of the spray head, guidewire, and/or tube surrounding the guidewire. Body passage devices are provided that allow the guidewire and/or spray head to be extended to universally adapt it to a variety of different length endoscope working channels or lumens. The body passage device containing a guidewire can be inserted into a working channel of an endoscope to clean, aspirate or irrigate the working channel or a certain patient tissue (e.g., colon, esophagus, etc.). This will allow removal of debris surrounding the endoscope and viewing the patient tissue will be clearer to better help diagnosis and treatment of the patient.

In one embodiment, there is a body passage device, comprising a housing configured to receive a guidewire, the housing having a longitudinal axis; a first guidewire adjuster contacting the housing and having a proximal end and a distal end, the first guidewire adjuster configured to extend the guidewire a first length along the longitudinal axis of the housing; and a second guidewire adjuster configured to contact the proximal end and/or the distal end of the first guidewire adjuster and/or guidewire and extend the guidewire a second length along the longitudinal axis of the housing.

In another embodiment, there is a body passage device, comprising an outer tube having proximal and distal ends and a longitudinal axis disposed therebetween, the outer tube having a channel therein extending along the longitudinal axis of the outer tube; a guidewire disposed in the channel and configured to extend through the proximal end of the outer tube and through and beyond the distal end of the outer tube; and a stabilizing member configured to prevent lateral and/or vertical movement of the guidewire relative to the channel of the outer tube.

In yet another embodiment, there is a body passage device, comprising a housing configured to receive a guidewire, the housing having a longitudinal axis; a first guidewire adjuster contacting the housing and having a proximal end and a distal end, the first guidewire adjuster configured to extend the guidewire a first length along the longitudinal axis of the housing; and a second guidewire adjuster configured to contact the proximal end and/or the distal end of the first guidewire adjuster and/or guidewire and extend the guidewire a second length along the longitudinal axis of the housing.

In another embodiment, there is a body passage device, comprising an outer tube having proximal and distal ends and a longitudinal axis disposed therebetween, the outer tube having a channel therein extending along the longitudinal axis of the outer tube; a guidewire disposed in the channel and configured to extend through the proximal end of the outer tube and through and beyond the distal end of the outer tube; and a stabilizing member configured to prevent lateral and/or vertical movement of the guidewire relative to the channel of the outer tube.

In one embodiment, in accordance with the principles of the present disclosure, a body passage cleaning device is provided. The device includes a tube including an inner surface defining a channel. An actuating element is movably disposed in the channel. The actuating element extends along a longitudinal axis between a first end and a second end. The second end includes a plug. A handle is coupled to the tube. The handle includes an inner surface defining a slot. The slot has an internal slider movably disposed therein such that the first end engages the internal slider. The internal slider comprises an inner surface defining a hole extending perpendicular to the longitudinal axis. At least a portion of the first end is disposed in the hole.

In one embodiment, in accordance with the principles of the present disclosure, the includes an outer tube having an inner surface defining a first channel. An inner tube is disposed the first channel. The inner tube includes an inner surface defining a second channel. An element is movably disposed in the second channel. The actuating element includes a first end a second end including a plug. The device comprises a plurality of ribs connecting the inner surface of the outer tube with an outer surface of the inner tube to fix the inner tube relative to outer tube such that the inner tube is coaxial with the outer tube.

In one embodiment, in accordance with the principles of the present disclosure, the device includes an outer tube including an inner surface defining a first channel. An inner tube is disposed in the first channel. The inner tube includes an inner surface defining a second channel. A plurality of ribs extending from the inner surface of the outer tube engage an outer surface of the inner tube to position the inner tube relative to the outer tube such that the inner tube is coaxial with the outer tube. An actuating element is movably disposed in the second channel. The actuating element includes a first end and a second end including a plug. A handle is coupled to the inner tube. The handle includes an inner surface defining a slot. The slot has an internal slider movably disposed therein such that the first end engages the internal slider. The internal slider includes an inner surface defining a hole extending perpendicular to the longitudinal axis. At least a portion of the first end is disposed in the hole.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 is a side, breakaway view of an embodiment of a device in accordance with the principles of the present disclosure;

FIG. 1A is a side view, in part cross section of components of an embodiment of the body passage device;

FIG. 1B is a side view, in part cross section of components of an embodiment of the body passage device;

FIG. 2 is a side, breakaway view of an embodiment of a device in accordance with the principles of the present disclosure;

FIG. 2A is a side view, in part cross section of components of the device shown in FIG.

FIG. 3 is a side view of components of the device shown in FIG. 2;

FIG. 4A is a cross sectional view of components of the device shown in FIG. 2;

FIG. 4B is a cross sectional view of components of the device shown in FIG. 2;

FIG. 5 is a side view of components of one embodiment of the device shown in FIG. 2, in accordance with the principles of the present disclosure;

FIG. 6 is a cross sectional view of components of the device shown in FIG. 5;

FIG. 7 is a cross sectional view of components of one embodiment of the device shown in FIG. 2, in accordance with the principles of the present disclosure;

FIG. 8 is a cross sectional view of components of one embodiment of the device shown in FIG. 2, in accordance with the principles of the present disclosure;

FIG. 9 is a cross sectional view of components of one embodiment of the device shown in FIG. 2, in accordance with the principles of the present disclosure;

FIG. 10 is a cross sectional view of components of the device shown in FIG. 9;

FIG. 11 is a cross sectional view of components of one embodiment of the device shown in FIG. 2, in accordance with the principles of the present disclosure; and

FIG. 12 is a cross sectional view of components of the device shown in FIG. 11.

Like reference numerals indicate similar parts throughout the figures. It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

New body passage devices and methods are provided for irrigating and/or cleaning the working channel of an endoscope or patient tissue that allows precision irrigation, aspiration, and/or cleaning by limiting unwanted lateral and/or vertical movement of the spray head, guidewire, and/or tube surrounding the guidewire. Body passage devices are provided that allow the guidewire and/or spray head to be extended to universally adapt it to a variety of different length endoscope working channels or lumens. The body passage device containing a guidewire can be inserted into a working channel of an endoscope to clean or irrigate the working channel a certain patient tissue (e.g., colon, esophagus, etc.). This will allow removal of debris surrounding the endoscope and viewing the patient tissue will be clearer to better help diagnosis and treatment of the patient.

The exemplary embodiments of the body passage cleaning device and related methods of use disclosed are discussed in terms of a device for cleaning a lumen of a colon or other body cavity, and more particularly to a device that limits lateral movement between a relatively small diameter elongate device and a larger diameter conduit in which the inner, small diameter elongate device is disposed to precisely control positioning of a spray head positioned at a distal end of the inner, small diameter elongate device and/or that provides an extra range of motion for the spray head using controls in the handle of the device.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

The following discussion includes a description of a body passage cleaning device and related methods of employing the body passage cleaning device in accordance with the principles of the present disclosure. Alternate embodiments are also disclosed. Reference is made in detail to the exemplary embodiments of the present disclosure, which are illustrated in the accompanying figures.

The device and methods provided limits lateral movement between a relatively small diameter elongate device and a larger diameter conduit in which the inner, small diameter elongate device is disposed to precisely control positioning of a spray head positioned at a distal end of the inner, small diameter elongate device and/or that provides an extra range of motion for the spray head using controls in a handle of the device.

When a relatively small diameter elongate device is passed through a larger diameter conduit, such as, for example, a guidewire within a partial-length conduit or a partial-length conduit within an outer conduit, there is a certain degree of freedom of lateral movement. Thus, the inner, small-diameter elongate device may be disposed, at certain points along its length, in the center of the larger diameter conduit, and at other points to be disposed closer to the inner wall of the larger diameter conduit. This degree of freedom of lateral movement will influence the overall distance between the two extremities of the inner, small-diameter device, when measured along the longitudinal axis of the large diameter conduit.

As a result of these degrees of freedom created when the relatively-small diameter elongate device is passed through the larger diameter conduit, moving a proximal end of the inner, small-diameter elongate device 1 cm, for example, may result in a longitudinal movement of less than 1 cm of a component disposed at a distal end of the inner, small-diameter elongate device, such as, for example, a distal spray head. The freedom of movement of the component disposed at a distal end of the inner, small-diameter elongate device may therefore reduce the precision with which the component disposed at a distal end of the inner, small-diameter elongate device may be positioned relative to another component of the device, another device, or a portion of a patient's anatomy. Consequently, a need exists for reducing the freedom of movement of the component disposed at a distal end of the inner, small-diameter elongate device as much as possible, in order to improve the precision with which an operator may control the position of the component disposed at a distal end of the inner, small-diameter elongate device.

Furthermore, some medical devices and/or systems may include a plurality of larger diameter conduits, such as, for example, endoscopes, each configured for movable disposal of a relatively small diameter elongate device, such as, for example, a guidewire. There are often differences (in the order of about 1-15 mm) in the overall lengths of the larger diameter conduits. Such differences may make placing a component disposed at a distal end of the inner, small-diameter elongate device, such as, for example, a distal spray head, at a desired position.

Attempts have been made to solve these abovementioned problems by adding more range of travel to a proximal handle mechanism to allow the proximal end of the inner, small-diameter elongate device to move over a longer distance. However, this solution may be problematic, since the device is intended to be held in one hand, and the additional length of the proximal handle would prevent the same. This disclosure describes an improvement over these prior art technologies including those body passage devices described in PCT/IL2009/000346 filed on Mar. 26, 2009, U.S. application Ser. No. 12/923,796 filed Oct. 7, 2010 and PCT/IL2011/000086 filed Jan. 11, 2011. These entire disclosures are herein incorporated by reference into the present

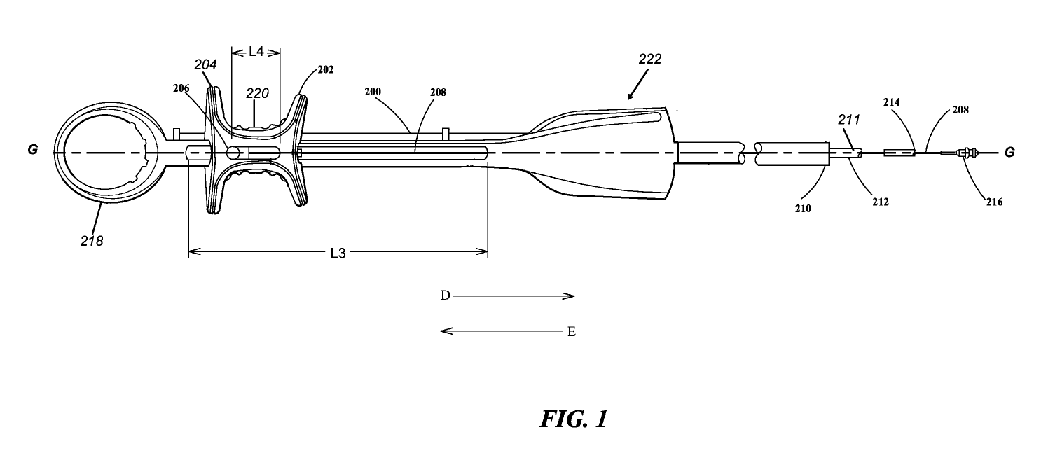

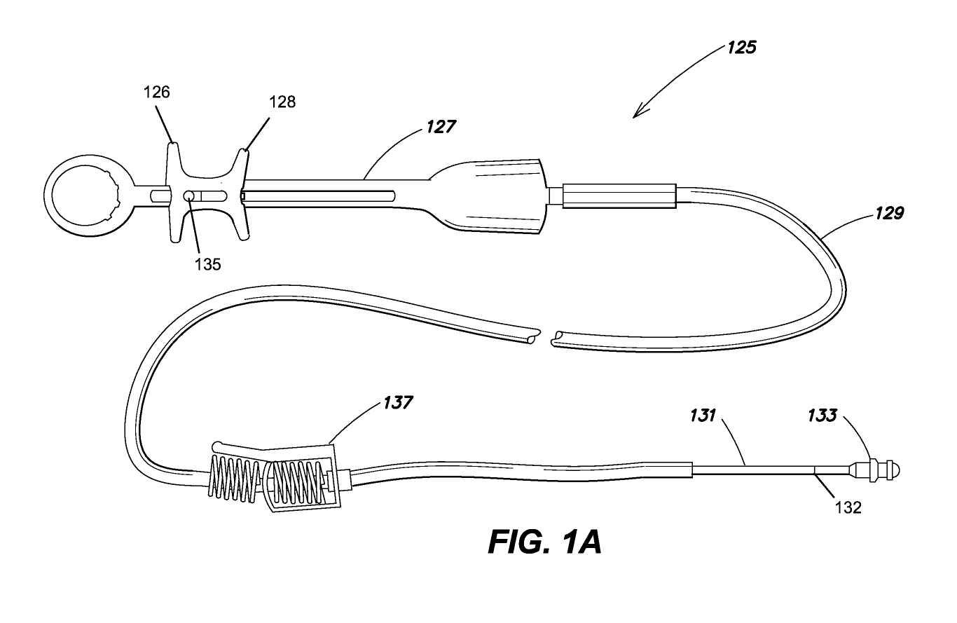

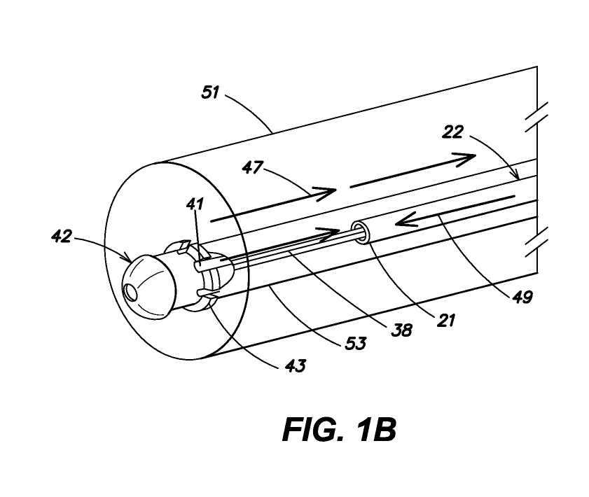

Turning to FIGS. 1 and 1A there are illustrated components of a body passage cleaning device. The body passage device 222 comprises a housing 200 configured to receive a guidewire 208, the housing 200 having a longitudinal axis G; a first guidewire adjuster 220 is shown contacting the housing and having a proximal end 204 and a distal end 202, the first guidewire adjuster configured to extend the guidewire 208 a first length shown as L3 along the longitudinal axis G of the housing; and a second guidewire adjuster 206 configured to contact the proximal and/or the distal end of the first guidewire adjuster and/or guidewire, and extend the guidewire a second length L4 relative to the first guidewire adjuster 220 along the longitudinal axis of the housing. The first guidewire adjuster 220 is shown as a slider that the user can place their thumb through thumb ring 218, which aids in moving slider and guidewire forward (shown as direction arrow D away from the thumb ring) and backward (shown as direction arrow E toward the thumb ring). In this way, guide wire 208 and spray head (or plug) 216 can be precisely manipulated in and out of the endoscope working channel or proximal or distal to patient tissue or a body cavity (e.g., colon, esophagus, etc.) so that the target area can be cleaned, aspirated, or irrigated with a fluid (e.g., sterile water, saline, normal saline, dextrose, etc.). Fluid will travel out spray head In some embodiments, spray head 216 can have deformable channels (not shown) that guide out of the working channel of the endoscope. When the channels are open, water is guided to the specific target area. When the channels are closed, from for example, the collapse of the on themselves when the spray head is inside the working channel of the endoscope, the spray 216 will also function as a deformable plug and prevent the fluid from going beyond the spray head. In this way, debris or other material inside the working channel of the endoscope can be cleaned or irrigated as well. The guide wire passes through the channel of outer tube 210. In embodiments, outer tube 210 can be a partial length outer tube and not run continuously with the guidewire as shown in the current embodiment.

If additional length is needed for the guidewire and/or spray head 216, due to, for example, the various lengths of endoscope working channels, including those that are the same makes and models, or if additional length is needed to irrigate or clean patient tissue, the user moves or slides second guidewire adjuster 206 in the direction shown in arrow D or forward to cause guidewire 208 and spray head 216 to move forward. Therefore, precise irrigation or cleaning can be achieved if needed, when water passes out of outer tube 210 and the stream is guided by spray head 216 to the target area. It will be understood that the first guidewire adjuster 220 or the second guidewire adjuster 206 can be moved forward as shown in arrow D or backward shown in arrow E to control the guidewire 208 and/or spray head 216. In this way, precise control of the cleaning and/or irrigation can be achieved.

In the embodiment shown, in FIG. 1, the second guidewire adjuster 206 is disposed within the first guidewire adjuster 220 and the first guidewire adjuster is slidably mounted along the longitudinal axis of the housing to extend the guidewire the first length. The one end of the guidewire 208 can directly contact the first guidewire adjuster 220 or in the embodiment shown, the guidewire 208 can be anchored at one end of the second guidewire adjuster 206 and the first guidewire adjuster does not directly contact the guidewire 208, but directly contacts second guidewire adjuster 206, which directly contacts guidewire 208 so that movement (e.g., sliding) of the first guidewire adjuster along the longitudinal axis of the housing 222 in the forward D, will also cause second guidewire adjuster to also move in direction D and thus guidewire 208 and spray head 216 will be moved further along longitudinal axis G. If additional length L4 is needed for the guidewire 208 due to, for example; using an endoscope with a different length working channel, the user will move or slide first guidewire adjuster 220 forward to length L3, then second guidewire adjuster 206 can further be moved or slid to the additional length L4 to adjust the guidewire 208 and spray head 216 this additional length. The guidewire adjusters can universally configured for both right and left hand users. The first guidewire adjuster 220 can be smaller in length and easier to manipulate based on the user's hand and finger size, as the second guidewire adjuster is available, if additional length is needed. In this way, the first and second guidewire adjusters can fit the user's hands comfortably.

In the embodiments, shown in FIG. 1, the first guidewire adjuster 220 and the second guidewire adjuster 206 are slidably mounted on the housing 222. However, other mountings are possible. It should also be understood that the first guidewire adjuster 200 can engage the guidewire until it is slid or moved all or part of the first length L3, then the second guidewire adjuster 206 can engage the guidewire to move or slide it all or part of the additional length or the second length L4. In this way, the guidewire and/or the spray head can be moved into discrete positions and the user can comfortably slide or move the guidewire to those positions.

In the embodiment shown in FIG. 1, the second guidewire adjuster 206 is disposed within the first guidewire adjuster 220 and parallel or substantially parallel to it so that moving one will move the other. However, it will be understood by those skilled in the art that other configurations are possible and the first guidewire adjuster can be orthogonal or perpendicular to the second guidewire adjuster. In the embodiment shown in FIG. 1, the guidewire 208 is anchored in the second guidewire adjuster 206 and the first guidewire adjuster's proximal end 206, does not directly contact the guidewire 208. In other embodiments, both the first guidewire adjuster and the second guidewire adjuster can both contact the guidewire.

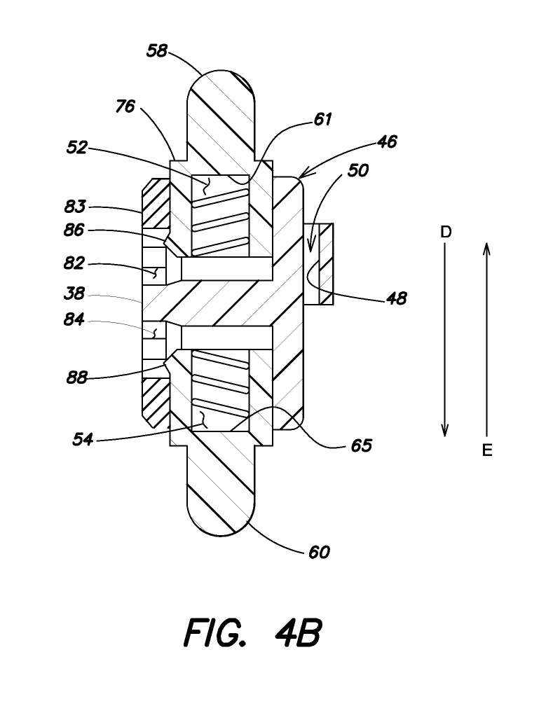

In some embodiments, the first guidewire adjuster and/or second guidewire adjuster can have a lock to hold it in a discrete position. These locking mechanisms can be a spring, button, or knob as shown in FIGS. 4A, 4B, and 6.

The body passage device, because portions of it have a smaller diameter than the diameter of the working channel of the endoscope, often at times the guidewire and the spray head have unwanted lateral and vertical motion, which may lead to imprecise cleaning and/or

To reduce or eliminated, this unwanted lateral and/or vertical movement, in some embodiments, the body passage device comprises a stabilizing member. More particularly, the body passage device has an outer tube 210 having proximal end in the direction of the first guidewire adjuster 220 and distal end in the direction of the spray head 216 and a longitudinal disposed therebetween, the outer tube 210 has a channel 211 therein extending along the longitudinal axis of the outer tube; a guidewire disposed 208 in the channel 211, which is configured to extend through the proximal end of the outer tube and through and beyond the end of the outer tube; and a stabilizing member shown as an inner tube 212 is configured to lateral and/or vertical movement of the guidewire relative to the channel of the outer tube. By placing an inner tube 212 coaxially with the outer tube 210, which may be a full length or partial length with the outer tube 210, or extend more than the length of the outer tube 210, the inner 212 increases the diameter of the device in the working channel and stabilizes the guidewire 208 and spray head 216 to reduce unwanted lateral and/or vertical movement of the guidewire. It be understood that the inner tube 212 can be disposed within the outer tube channel 211.

In some embodiments, the inner tube 212 is the same length as the outer tube 210. In some embodiments, the inner tube 212 is a smaller length than the outer tube 210. In some embodiments, the inner tube 212 is larger than the length of the outer tube 210. In some embodiments, the inner tube 212 extends further along the longitudinal axis G than the outer tube as shown in FIG. 1.

In some embodiments, in addition to the inner tube 212 or in place of it, the body passage device can have a different stabilizing member that comprises a ring, rib, or spring disposed in the channel 211 of the outer tube 210, which is configured to contact the guidewire to reduce lateral and/or vertical movement of the guidewire. In some embodiments, the ring, rib, or spring; can directly contact the guidewire 208 to reduce or eliminate unwanted lateral and/or vertical movement of the guidewire. In some embodiments, the ring, rib, or spring can contact inner tube 212 and/or outer tube 210 to reduce or eliminate lateral and/or vertical movement of the guidewire. For example, the stabilizing member 214 can be disposed within channel 211 of the outer tube and contact outer tube and the guidewire 208 to reduce or eliminate lateral and/or vertical movement of the guidewire.

In another embodiment, shown in FIG. 1A, there is a body passage device 125 comprising a housing 127 configured to receive a guidewire, the housing 127 has a longitudinal axis, and a first guidewire adjuster is shown contacting the housing and having a proximal end and a distal end 128, the first guidewire adjuster configured to extend the guidewire a first length shown along the longitudinal axis of the housing; and a second guidewire adjuster 135, which is configured to contact the proximal end and/or the distal end of the first guidewire adjuster and/or guidewire and extend the guidewire a second length relative to the first guidewire adjuster along the longitudinal axis of the housing. The first guidewire adjuster is shown as a slider that the can place their thumb through the thumb ring, which aids in moving slider and guidewire or backward. In this way, guide wire and spray head (or plug) 133 can be precisely manipulated and out of the endoscope working channel or proximal or distal to patient tissue or a body cavity (e.g., colon, esophagus, etc.) so that the target area can be cleaned or irrigated with a fluid (e.g., sterile water, saline, normal saline, dextrose, etc.). Fluid will travel out spray head 133. In some embodiments, spray head 133 can have deformable channels (not shown) that guide water out of the working channel of the endoscope. When the channels are open, water is guided to the target area. When the channels are closed, from for example, the collapse of the channels on themselves when the spray head is inside the working channel of the endoscope, the spray head will also function as a deformable plug and prevent the fluid from going beyond the spray head. this way, debris or other material inside the working channel of the endoscope can be cleaned or irrigated as well. The guide wire passes through the channel of outer tube 129. In some embodiments, outer tube 129 can be a partial length outer tube and not run continuously with the guidewire as shown in the current embodiment.

To reduce or eliminated, this unwanted lateral and/or vertical movement, in some embodiments, the body passage device comprises a stabilizing member. More particularly, the body passage device has an outer tube 129 having proximal end in the direction of the second guidewire adjuster 135 and distal end in the direction of the spray head 133 and a longitudinal axis disposed therebetween, the outer tube 129 has a channel therein extending along the longitudinal axis of the outer tube; a guidewire disposed in the channel, which is configured to extend through the proximal end of the outer tube and through and beyond the distal end of the outer tube; and a stabilizing member shown as an inner tube 131 is configured to prevent lateral and/or vertical movement of the guidewire relative to the channel of the outer tube.

By placing an inner tube 131 coaxial with the outer tube 129, which may be a full length or partial length with the outer tube 129, or extend more than the length of the outer tube 129, the inner tube 131 increases the diameter of the device in the working channel and stabilizes the guidewire and spray head 131 to reduce unwanted lateral and/or vertical movement of the guidewire. The inner tube 131 can be disposed within the channel of outer tube 129.

In some embodiments, in addition to the inner tube 131 or in place of it, the body device can have a different stabilizing member that comprises a ring, rib, or spring 132 disposed the channel of the inner tube 131, which is configured to contact the guidewire to reduce lateral and/or vertical movement of the guidewire. In some embodiments, the ring, rib, or spring; can directly contact the guidewire to reduce or eliminate unwanted lateral and/or vertical movement the guidewire. In some embodiments, the ring, rib, or spring can contact inner tube 131 and/or outer tube 129 to reduce or eliminate lateral and/or vertical movement of the guidewire.

In the embodiment shown in FIG. 1A, the body passage device comprises a valve or clamp 137 to shut off water flow entering the inner and/or outer tube. The portions of the device containing the guidewire, inner and/or outer tube can be inserted into a working channel of an endoscope to clean or irrigate the working channel or patient tissue.

In the embodiment shown in FIG. 1B, the outer tube 22, stabilizing member 21, guide wire 38 are disposed in the working channel 53 of an endoscope 51. In the embodiment shown, the spray head 43 and plug 42 are deformable (e.g., they are made from, for example, elastomeric material) and they are shown being retracted into the working channel 53 of the endoscope 51. This can be accomplished by moving the first guidewire adjuster (220 of FIG. 1) and/or second guidewire adjuster (206 of FIG. 1) in the direction of arrow E of FIG. 1. In this way, guide wire 38 and spray head 43 and plug 42 can be precisely manipulated in and out of the endoscope working channel so that the target area can be cleaned or irrigated with a fluid (e.g., sterile water, saline, normal saline, dextrose, etc.). The channels 41 of the spray head will deform or collapse as the spray head 43 and plug 42 are retracted into the working channel due to the fact that the plug, spray head and/or channels will expand outside of the working channel 53. As the plug, spray head and/or channels retract into the working channel of the endoscope; the channels 41 of the spray head will contract or collapse or close on themselves due to the snug fit within the working channel and create a seal, which will prevent fluid flow in the working channel. In some embodiments, the plug 42 is also deformable, and will seal the working channel and prevent forward fluid flow 49 in the working channel beyond that of the channels 41 of the spray head. This will create back fluid pressure shown by arrow 47 that can irrigate and/or clean debris in the working channel.

In FIG. 1B, the stabilizing member 21 is shown as a ring which compresses on the guidewire 38 and reduces or eliminates unwanted lateral and/or vertical movement of the guidewire.

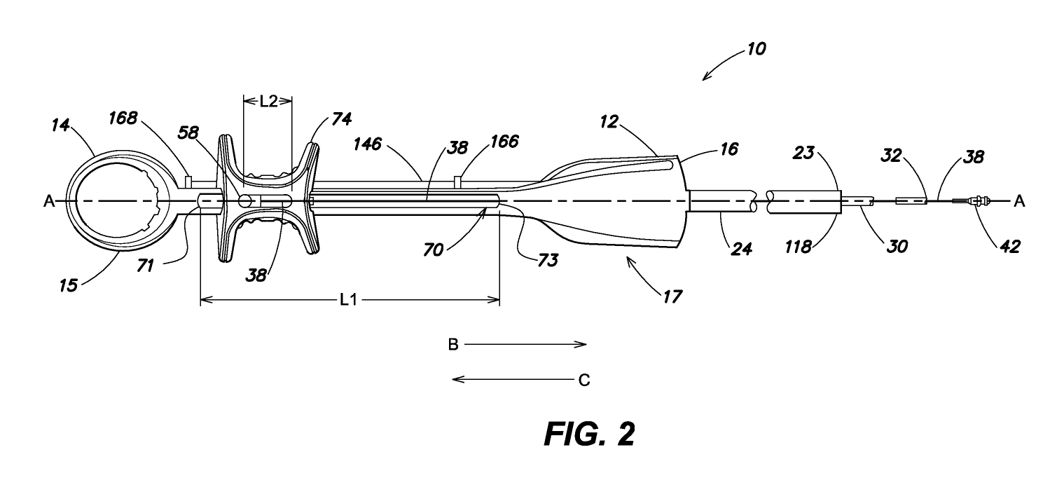

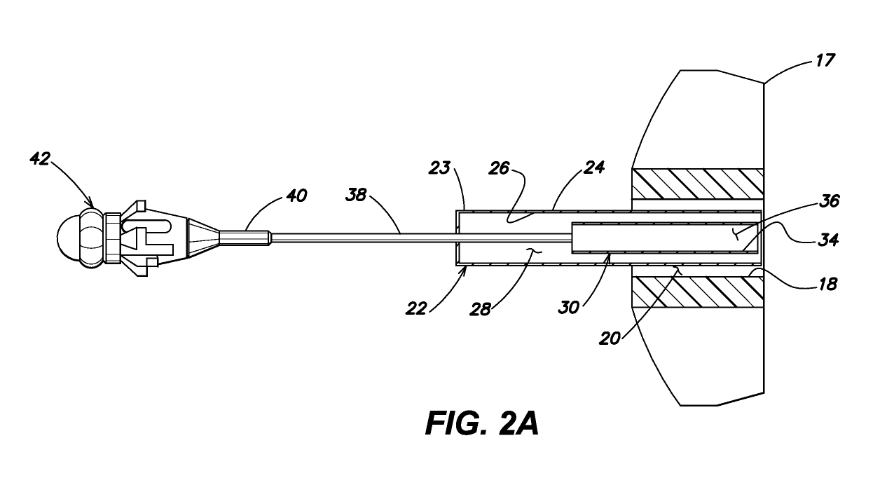

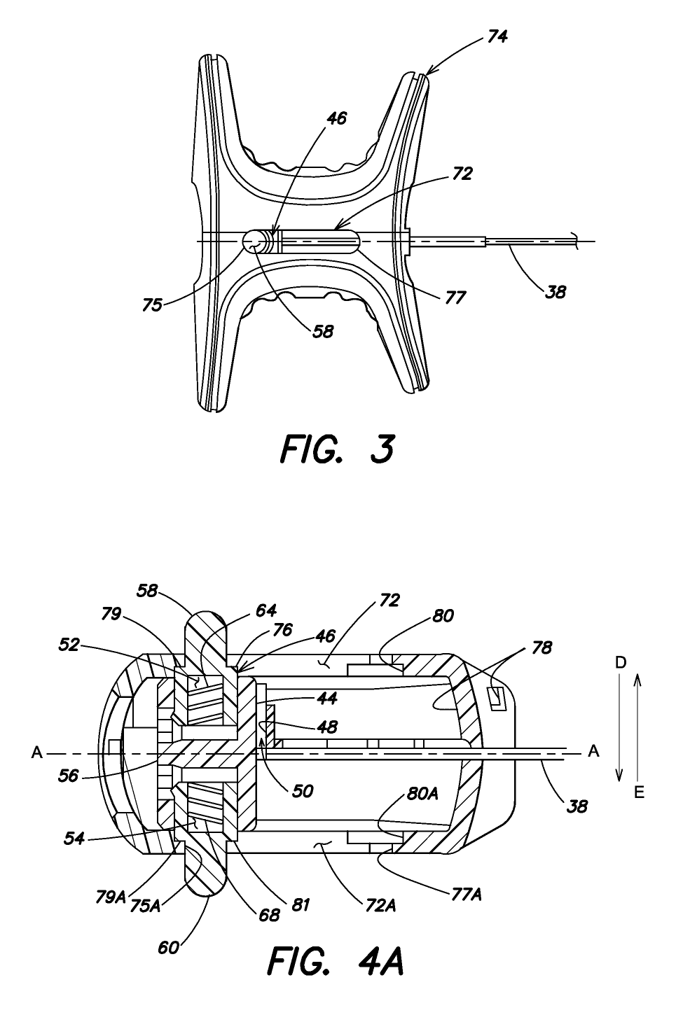

Turning to FIGS. 2-4B, there are illustrated components of a body passage cleaning 10. The components of device 10 can be fabricated from biologically acceptable materials for medical applications, including metals, synthetic polymers, ceramics, or composites. For example, the components of device 10, individually or collectively, can be fabricated from materials such as machined metal and/or machined or injection molded plastic, stainless steel alloys, commercially pure titanium, titanium alloys, Grade 5 titanium, super-elastic titanium alloys, cobalt-chrome alloys, stainless steel alloys, super-elastic metallic alloys (e.g., Nitinol, super elasto-plastic metals, such as GUM METAL® manufactured by Toyota Material Incorporated of Japan), ceramics, thermoplastics such as polyaryletherketone (PAEK) including polyetheretherketone (PEEK), polyetherketoneketone (PEKK) and polyetherketone (PEK), carbon-PEEK composites, PEEK-BaS04 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyetherimide, polyethylene, epoxy, partially resorbable materials, totally resorbable materials, polyglycolide, polytyrosine carbonate, polycaroplaetohe and their combinations, bioactive glasses, porous metals, or any combination thereof.

Various components of device 10 may have material composites, including the above materials, to achieve various desired characteristics such as strength, rigidity, elasticity, compliance, biomechanical performance, durability and radiolucency or imaging preference. The components of device 10, individually or collectively, may also be fabricated from a heterogeneous material such as a combination of two or more of the above-described materials. The components of device 10 may be monolithically formed, integrally connected or include fastening elements and/or instruments, as described herein.

Device 10 is configured to be introduced through the working channel of a medical device, such as, for example, an endoscope to cleanse a body cavity, such as, for example, the colon of a patient. Device 10 includes a housing, such as, for example, a handle 12 extending along a longitudinal axis A between an end 14 defining a thumb ring 15 and an end 16 opposite end 14 that defines a manifold 17. Handle 12 includes an inner surface 18 defining an elongated cavity 20. Handle 12 may be manufactured from ABS, polycarbonate, Delrin and other plastic resins depending on the compatibility with the sterilization method to be used (autoclaving, Gamma radiation or ETO, preferably, by means of casting in mass production. The length of handle 12 may generally be about 80-120 mm.

In some embodiments, handle 12 includes one or more passages for connecting a fluid-supply channel to one of two or more fluid outlet channels. In some embodiments, handle 12 includes means for switching between the fluid outlet channels to which said fluid-supply channel is connected. In some embodiments, handle 12 includes one fluid outlet channel in fluid communication with a passageway 36 of an inner tube 30, and a second fluid outlet channel in fluid communication with the space surrounding the external surface of tube 30. When inserted into a working channel of an endoscope, this latter space will be bounded externally by the walls of the working channel. In some embodiments, handle 12 may be used to operate endoscopic instruments, such as a colonic cleansing device of the present disclosure. In such embodiments, handle 12 incorporates a mechanism for directing flow to different lumens and conduits. It is to be emphasized that in addition to its use in conjunction with the colonic cleaning device incorporating tube 30, handle 12 may also be used together with variants of this device (e.g. not comprising an outer tube) and indeed with other endoscopic instruments. In some embodiments of handle 12, the aforementioned switching means comprises a multi-way fluid valve. In some embodiments, the coupling means comprises a mechanical actuator (for example in the form of an elongated strip or bar) which is connected to both a slider mechanism and to the aforementioned switching means. In some embodiments, tube 30 may be made from ETFE, PTFE or other plastic resins which are compatible with the applicable sterilization method with low friction coefficient and sufficient rigidity to support the device without collapsing. In some embodiments, tube 30 may be made of silicon or rubber resin, preferably from PTFE for low friction between the guidewire and tube as well as between tube 30 and the working channel, having a length of about 100 cm-210 cm, depending on the endoscope length used as well as the external extension tubing length, and having an inner diameter generally in the range of 1 mm preferably smaller to reduce hysteresis effects (around AWG16), and a wall thickness of about

Tube 22 is positioned in cavity 20 such that an outer surface 24 of tube 22 engages 18. Tube 22 enables device 10 to function either within a working channel of an endoscope (or other instrumental lumen) or within a naturally-occurring channel or passageway (such as the colonic lumen). Tube 22 provides device 10 with the following additional advantages: a) a stand alone configuration which permits the use of the device in any lumen (natural or instrumental) including endoscopic working channels; b) device 10 may extend beyond the distal end of the working channel and/or other natural or instrumental lumen; c) a camera may be included on 22 to enable viewing of further areas while performing the cleansing; d) aspiration and irrigation will be done through the gap between tube 22 and tube 30. In some embodiments, tube 22 may constructed such that it has a flexible, semi rigid or rigid configuration. In some embodiments, tube 22 may be made from ETFE, PTFE, and Nylon etc., preferably from PTFE, has a length of about 50-70 cm, depending on the endoscope length used as well as the external extension tubing length, an inner diameter generally similar to the working channel diameter in the range of 2 mm preferably smaller to reduce hysteresis effects (around AWG8), and wall thickness of about 0.5-1 mm.

Tube 22 is fixed relative to handle 12. An end 23 of tube 22 extends beyond manifold 17 such that end 23 is positioned outside of cavity 20. In some embodiments, tube 22 can be variously connected with handle 12, such as, for example, monolithic, integral connection, frictional engagement, threaded engagement, mutual grooves, screws, adhesive, or raised element. In some embodiments, tube 22 comprises a flexible material to allow tube 22 to bend during insertion into and/or translation through a lumen of a patient's body, such as, for example, the patient's colon. In some embodiments, tube 22 comprises a transparent or translucent material to facilitate viewing of components disposed within tube 22.

In some embodiments, tube 22 is constructed from a non-compliant material such as nylon, Pebax (or a blend thereof), polyurethane and polyethylene terephthalate (PET). In such a case, tube 22 has a random flat shape when in an unexpanded configuration, which becomes circular in cross section when expanded with irrigation fluid. In some embodiments, tube 22 is made from a compliant material such as silicone or a thermoplastic elastomer (TPE), wherein tube 22 is able to expand in the same manner as a compliant balloon. In some embodiments, tube 22 may be made from ETFE, PTFE, and Nylon etc., preferably from PTFE, having a length of about 50-70 cm, depending on the endoscope length used as well as the external extension tubing length, an inner diameter generally similar to the working channel diameter in the range of 2 mm-4 mm preferably smaller to reduce hysteresis effects (around AWG8), and wall thickness of about 0.5-1 mm.

In some embodiments, tube 22 includes a lumen extending parallel to passageway 28 running along the length of the tube 22. This additional lumen may be used for a number of purposes including: injection of therapeutic agents, injection of iodine (for chromo-endoscopy), application of very cold water in order to arrest bleeding, and delivery of tumor-specific bio-bio-markers. In addition, the extra lumen may be used to introduce air for insufflation of air mixture with the irrigation fluid. It is to be noted that the additional lumen may have a very diameter (e.g. 0.2-1 mm) and may be contained within a collapsible tube.

In some embodiments, tube 22 does not change its diameter under the liquid pressure (e.g., it is non-collapsible). Such a configuration may be advantageous when it is required mainly to irrigate a specific location without necessarily allocating more space to the working channel. This configuration may also be advantageously employed in situations wherein the space between the working channel and the lumen of the irrigation water is sufficiently large to allow aspiration and/or insertion of additional tooling through the working channel. This particular embodiment, while employing a conventional catheter tube is characterized by the following notable features: a) miniaturization of the spray head 42 to 2-3 mm overall diameter to enable insertion through small lumens such as the working channel of a colonoscope; b) the irrigation fluid is directed through channels or nozzles in spray head 42; and c) it is required to design specific nozzles to optimize the pressure flow and avoid turbulences wherever possible; d) enables maximum liquid momentum with minimal liquid volume. For example, if the irrigation liquid is transformed to a spray wherein the liquid drops are very small no cleansing effects can be achieved. Alternatively, if the nozzle is too large (e.g. 0.8 mm-3.8 mm) to provide the cleansing momentum required for irrigation an impractically-large volume of irrigation fluid will be required. The irrigation nozzles are focused forward (in an inward, outward angles or straight), thus enabling the physician maximum efficiency and maximum effective force (liquid momentum) in the direction where the colonoscope camera is directed. Automatic cleansing in 360° enables irrigation where no vision is available (for example, out of the FOV or in

An inner surface 34 of tube 30 defines a passageway 36 having a cylindrical cross sectional configuration configured for movable disposal of an actuating element, such as, for example, a guidewire 38. Guidewire 38 has a length that is greater than that of tube 30, such that portion of guidewire 38 is left unenclosed by tube 30. Guidewire 38 includes an end 40 defining spray head 42. Spray head 42 includes a plurality of channels, apertures and/or nozzles which capable of allowing the passage of a fluid therethrough. At least an outer portion of spray head is capable of being elastically deformed such that an external diameter thereof may be reduced in response to inwardly-directed compression forces exerted thereon. The channels, apertures nozzles of spray head 42 are in a closed conformation when spray head 42 is subject to compression forces, and are in an open conformation when spray head 42 is not subject to said compression forces such that the fluid is capable of passing through the channels, apertures nozzles when the channels, apertures and/or nozzles are in the open conformation and is of passing through the channels, apertures and/or nozzles when the channels, apertures and/or nozzles are in the closed conformation. Further details regarding spray head 42 can be found in PCT/IL2009/000346 filed on Mar. 26, 2009, U.S. application Ser. No. 12/923,796 filed Oct. 7, 2010 and PCT/IL2011/000086 filed Jan. 11, 2011, which are each incorporated herein by reference, in their entireties.

In some embodiments, guidewire 38 comprises a flexible material to allow guidewire 38 to bend during insertion into and/or translation through a lumen of a patient's body, such as, for example, the patient's colon. In some embodiments, guidewire 38 can contain one or more radiopaque materials at various points along its length (not shown). These radiopaque markers may be used to locate the device by means of real time visualization using X-ray imaging. The use of such markers is particularly important in upper GI endoscopic procedures, for example when introducing endoscopic tooling trough the papilla. In some embodiments, guidewire 38 has a diameter of about 0.25 mm to about 0.6 mm. In some embodiments, guidewire 38 has a diameter of about 0.3 mm to about 0.8 mm. In some embodiments, guidewire 38 has a diameter of about 0.4 mm to about 1.5 mm. In some embodiments, guidewire 38 is made from stainless steel 304V with an optional configuration of PTFE coating to reduce potential friction between guidewire 38 and tube 30 and its diameter may generally be in range of 0.5-0.7 mm, preferably

In some embodiments, passageway 36 defines a channel configured for both passing irrigation fluid distally and aspirating fluid and solid debris proximally, wherein both of these processes may be performed in a highly effective manner as part of a procedure for cleansing internal body passages and cavities. This configuration permits effective and higher-pressure irrigation of a body cavity via an endoscopic working channel while still allowing for the use of same working channel for other purposes, most particularly the aspiration of fluid and debris said body cavity. This configuration also permits irrigation and cleansing of the working channel of an endoscopic instrument without the removal of the instrument from the body, such that blockages of the channel by fecal material and debris may be prevented or removed. This configuration further permits irrigation, cleansing and aspiration for additional applications such as, for example, upper and lower GI bleeding, bronchoscopy, cystoscopy, gastrostomy trauma surgery where no preparation was available and endo-surgery preparation and additionally the upgrading of all endoscopic devices by integrating them together with a nozzle assembly, thereby not requiring the replacement of tools (for example biopsy forceps, snares, injection needles, and so on) during the procedure. In some embodiments, to pump irrigation fluid into passageway 36, tube 30 may be connected to a positive pressure liquid pump (centrifugal, peristaltic or other) or to a manual injector. To cause collapse of tube 30, it can be connected to a manual injector or through a negative pressure vacuum pump.

In some embodiments, tube 30 is used in an endoscope working channel (or similar instrumental lumen). An attachment seal is inserted around tube 30 at the proximal entrance to passageway 36. This sealing element, which may be constructed of a flexible material such as a biocompatible rubber, plastic or metal, prevents irrigation and debris being sprayed on the operator. In addition, its presence is necessary for maintaining integrity of the negative aspiration pressure that is applied to the space between tube 22 and tube 30. The presence of tube 30 in device 10 results in liquid and debris aspiration taking place through device 10 (in the space between tubes 22, 30) rather than through the working channel itself. This arrangement therefore requires the inclusion of a Y-connector at the proximal end of the working channel, in order to direct the aspirated liquid and solid material—via a one-way valve—along a waste line to a collection container.

In some embodiments, tube 30 comprises a collapsible sheath. A proximal end of tube is connected to a source of irrigation fluid (e.g. saline) and suitable pumping apparatus. When irrigation fluid is pumped through passageway 36, the tube 30 moves from a flaccid, collapsed state to a fully expanded conformation, thereby allowing maximum transfer of the irrigation fluid to a spray head 42, details of which are discussed hereinbelow. When the irrigation fluid ceases flow through tube 30 (e.g. as a result of both turning off the irrigation fluid pump and connecting the proximal end of tube 30 to a negative pressure source), tube 30 loses its source of structural rigidity (the column of irrigation fluid) and returns to the flaccid, collapsed state, thereby increasing the volume of a working channel that is external to the irrigation catheter. This is advantageous, for at least the following three reasons: a) maximum space for aspiration of irrigation fluid and fecal debris through the working channel is provided; b) additional space for the introduction and passage of endoscopic surgical tools (without the need to remove the irrigation catheter) is created; c) insufflation of the body cavity (e.g. colon) may be performed in the presence of tube 30, when collapsed. In some embodiments, irrigation fluid is pumped passageway 36 under positive pressure, said fluid is directed to the body cavity that is being cleansed (e.g. the colon). Conversely, when a negative pressure source is connected to the proximal end of tube 30, tube 30 collapses, thereby creating a larger free volume within passageway 36. In order to create optimal sheath collapse, a one way valve may be used.

It should be noted that for the purpose of the present disclosure the term “distal spray head unit” and the like are sometimes used interchangeably with the terms “distal plug” or “spray head.” It should further be noted that the term “distal” refers to the direction away from the operator and towards the center of the patient's body. Consequently, the term “proximal” is taken to refer to the opposite direction. In some embodiments, the external diameter of spray head 42 when not subjected to inwardly-directed radial compression forces is slightly larger than the internal diameter of passageway 36. In some embodiments, the internal diameter of passageway 36 is 3.8 mm, and generally in the range of 2-4 mm.

Since the distal-proximal location of guidewire 38 is altered during use, while the position of tube 30 is fixed (with reference handle 12), the precise distance between the distal end of tube 30 and the more distally placed distal end of guidewire 38 will also alter, and will generally be in the range of about 1 cm to about 4 cm. In general, the total length of guidewire 38 (which is typically constructed of 0.5-0.6 mm diameter) will be in the range of about 150 cm-210 cm, depending on the endoscope length used as well as the external extension tubing length. Extension tubing is assembled between the endoscope working channel adapter and the handheld device, typically having a length of 50-70 cm with an internal diameter of similar to the working channel diameter (commonly 3.8 mm). In some embodiments, the distal end of guidewire 38 is attached to the distal spray head unit by means of gluing, bonding or laser welding/soldering of the metal guide wire and the distal plug. Tube 30 is constructed of PTFE tubing (for low friction) or ETFE, depending on the sterilization method of the device to be used, and has an external diameter of about 1-mm-1.6 mm and a wall thickness of about 0.25 mm. It is to be recognized, however, that these measurements are given only as a general guide and do not limit the scope of the present disclosure in any way.

In some embodiments, spray head 42 is constructed such that it may be caused to move between two conformations. In the first conformation, wherein spray head 42 has a size that permits its distal passage through passageway 28 prior to irrigation and aspiration, and its passage following the end of those procedures. This conformation is also used for sealing the distal exit of passageway 36, such that it may assist in cleansing the endoscope working channel utilizing tube 30 (the distal end of which ends a few centimeters from the distal end of guidewire 38) to supply high pressure irrigation fluid to the distal part of passageway 36. In this way, hydrostatic pressure forces are added to the vacuum pressure, thereby significantly increasing the efficiency with which particulate matter may be moved proximally from the distal end of passageway 36 and thus preventing and/or clearing blockages therein. Generally, this first conformation is adopted when device 10 is contained within the confines of the passageway 36 (other narrow instrument channel). The external diameter of spray head 42 is generally constructed to be only slightly larger (by a few millimeters) than the internal diameter of passageway 36, such that when spray head 42 is contained within passageway 36, its outer diameter becomes reduced, and the previously open channels and apertures in the plug become closed. In the second conformation, spray head 42 has larger external dimensions than in the position. This occurs when spray head 42 leaves the confines of passageway 36 (at its distal A proximally-directed force is then applied such that spray head 42 makes contact with the distal exit of passageway 36, effectively providing a fluid seal over said exit, such that the only fluid transfer between passageway 36 and the region of the body cavity located beyond the distal end thereof is by way of channels, apertures and/or nozzles formed within spray head 42. In some embodiments, the first and second conformations are incorporated into a single spray head 42.

Spray head 42 is capable of being moved between the various above-described conformations by virtue of possessing one or more structural or functional features that permit the dimensions of spray head 42 to be altered by the operator, for example, by using linear shift of spray head 42 from inside the endoscope working channel to outside and vice versa. Device 10, is not, however, limited to such a mechanism but rather encompasses further embodiments that may include other mechanical mechanism or inflatable mechanisms that can be used to alter the spray head conformation and the use of flexible resin silicone or rubbers.

Spray head 42 effectively functions as a perforated plug that may be caused to partially or completely block the distal exit of passageway 36. Thus, when partially blocking said distal exit, irrigation fluid is supplied through passageway 36, the fluid being caused to exit spray head 42 at a higher pressure, in the form of a jet scatter directed towards the region of the body cavity (e.g., colon) located immediately distal to the distal end of passageway 36.

The irrigation fluid may be supplied to spray head 42 in the following manner: the irrigation fluids fed into passageway 36 using a positive pressure water pump (peristaltic, centrifugal pump, dosing pump, gearwheel pump, etc.) at a pump outlet pressure of between 2 and 10 atmospheres, resulting in a pressure range of 2-8 atmospheres in the outlet nozzle. The flow rate may range between 0.2 and 2 l/min. Sealing elements, adapters and connectors using standard Luer components may be used. It is to be emphasized that the abovementioned pressure and flow parameters are for the purpose of illustration only.

In use, spray head 42, while in its second, expanded conformation may be moved distally from its seated position over the exit of passageway 36 (as described immediately hereinabove) such that free fluid transfer between passageway 36 and the region of the body cavity located beyond the distal end thereof is once again possible. In this state, passageway 36 may be employed as a suction channel for the aspiration of fluid and solid debris from the body cavity, as well as for the passage of endoscopic tools.

It is to be emphasized that the potential use of passageway 36 as an aspiration channel is facilitated by the fact that spray head 42 is mounted on the aforementioned very small diameter guidewire 38 made of either metal or plastic resin), rather than on a fluid-supply catheter that would occupy a correspondingly larger fraction of the available working channel volume. Guidewire 38 is sufficiently rigid that it allows the operator to advance spray head 42 through passageway 36 and out into the body passage lumen. However, it also needs to be sufficiently flexible in order to negotiate bends and convolutions within said body passage.

In some embodiments, spray head 42 comprises an external O-ring constructed of a flexible material such as silicone. The presence of this O-ring assists in providing a smooth transition between the various head unit positions when moving from one operational mode to another. The use of such an O-ring is of particular value when device 10 is used in conjunction with an endoscope that has an internal taper at the distal end of the working channel. In such a case, the passage of spray head 42 through the working channel will largely be friction-free until spray head 42 enters the narrowed distal portion of the working channel.

Device 10 may be used for irrigation and cleansing of the working channel of an endoscopic instrument, in a manner such that blockages of said channel by fecal material may be prevented or removed. Thus, whenever the working channel becomes blocked by feces (and/or other solid and semi-solid material), or alternatively before it is thus blocked, spray head 42 is mounted on a flexible wire, such as, for example, guidewire 38, which in turn passes through a hollow tube, such as, for example, an inner tube 30, through which irrigation fluid can pass. 30 may have side apertures formed along its entire length or a portion thereof and/or an aperture the distal end.

In addition to the fluid causing reduction in the size of the solid debris an additional usage of the fluid is to create a positive hydrostatic pressure force (e.g. between 3 and 8 atmospheres) in order to help push the feces and blockage backwards, in addition to the vacuum force which is limited to a maximum of −1 atmosphere of pressure. In practice, the vacuum pressure actually achieved at the distal part of the endoscope may be much less than −1 atm. In this configuration it is necessary to seal the distal end of the endoscope.

In some embodiments, device 10 includes means for assisting an operator to recognize and detect the position of spray head 42 during use. In one such embodiment, one half of a ratchet mechanism is fitted to the distal face of the endoscope adjoining the distal exit of passageway 36. A complementary ratchet surface is incorporated into the proximal face of spray head 42 such that when spray head 42 is brought into contact with the distal exit of passageway 36, a clicking sound (caused by the ratchet mechanism) is emitted, thus informing the operator that the spray head 42 is in close apposition with the endoscope distal face. Other embodiments incorporate different mechanisms for signaling the position spray head 42, including remote sensors or transmitters located on the distal face of the endoscope that communicate with receivers or transmitters located on handle 12.

An end 44 of guidewire 38 opposite end 40 engages a slider 46 that is movably disposed within cavity 20, as shown in FIG. 4A. Slider 46 includes an inner surface 48 defining an 50 extending perpendicular to axis A configured for disposal of end 44. As such, end 44 is bent such that end 44 is perpendicular to end 40. In some embodiments, end 44 can be variously connected with slider 46, such as, for example, monolithic, integral connection, frictional engagement, threaded engagement, mutual grooves, screws, adhesive, nails, barbs and/or raised element. In some embodiments, aperture 50 and/or end 44 may be disposed at alternate orientations, relative to axis A and/or end 40, such as, for example, transverse, perpendicular and/or other angular orientations such as acute or obtuse, co-axial and/or may be offset or staggered. In some embodiments, end 44 may have various surface configurations, such as, for example, smooth and/or surface configurations to enhance fixation with aperture 50, such as, for example, rough, arcuate, undulating, dimpled, polished and/or textured.

Slider 46 includes an inner surface defining a recess 52 extending perpendicular to axis A. An inner surface of inner slider 46 defines a recess 54 extending perpendicular to axis A positioned opposite recess 52. Recesses 52, 54 are separated by a wall 56 of inner slider 46 that extends parallel to axis A. Recesses 52, 54 are spaced apart from aperture 50. Slider 46 includes a button 58 movably disposed in recess 52 and a button 60 movably disposed in recess 54. Button 58 includes an inner surface 61 defining an indent 62 configured for disposal of a spring 64 such that spring 64 engages wall 56 and surface 61 opposite wall 56. Spring 64 is biased to move button 58 in the direction shown by arrow E. A force may be applied to button 58 to overcome the force provided by spring 64 to allow button 58 to move in the direction shown by arrow D. Button 60 includes an inner surface 65 defining an indent 66 configured for disposal of a spring 68 such that spring 68 engages wall 56 and surface 65 opposite wall 56. Spring 68 is biased to move button 60 in the direction shown by arrow D. A force may be applied to button 60 to overcome the force provided by spring 68 to allow button 60 to move in the direction shown by arrow E. In some embodiments, recess 52, recess 54 and/or wall 56 may be disposed at alternate orientations, relative to axis A and/or end 40, such as, for example, transverse, perpendicular, parallel and/or other angular orientations such as acute or obtuse, co-axial and/or

Button 58 extends through a groove 70 in handle 12 and a groove 72 in an external slider 74 that is movable along handle 12 to move slider 46 in the direction shown by arrow B or the direction shown by arrow C. Groove 70 has a length L1 defined by a distance between an end 71 of groove 70 and an end 73 of groove 70 opposite end 71. Groove 72 has a length L2 defined by a distance between an end 75 of groove 72 and an end 77 of groove 72 opposite end 75 that is less than length L1. Button 60 extends through a groove 70A (not shown) in handle 12 and a groove 72A in slider 74. Groove 70A is aligned with groove 70 and has a configuration similar to that of groove 70. Groove 72A is aligned with groove 72 and has a configuration similar to that of groove 72. Groove 70A has a length L1. Groove 72A has a length L2 defined by a distance between an end 75A of groove 72A and an end 77A of groove 72A opposite end 75A. Slider 74 is movable within grooves 70, 70A in the directions shown by arrows B and C between ends 71, 73. The distance in which end 40 may be moved in order to alter the overall length of guidewire 38 is thus length L1.

In some embodiments, slider 74 is movable along a rod 146 that is fixed to handle. Rod 146 includes a distal stop element 166 and a proximal stop element 168, stop elements 166, 168 being designed to be respectively pushed and pulled by slider 74. A distal end of rod 146 is fitted with a pair of jaws (not shown) which are used to grasp the movable end of valve lever 150. Thus, when external slider 152 is pushed distally, a movable end of a valve lever within the water box is similarly moved in a distal direction. Conversely, when slider 74 is pulled by the operator in a proximal direction, the movable end of the valve lever is rotated in a counter-clockwise direction, and thereby moved into its proximal position. It should also be noted that since guidewire 38 is connected to slider 74 via slider 46, movement of slider 74 distally or proximally will respectively cause distal advancement or proximal retreat of spray head 42 that is connected to guidewire 38. In this way, movement of slider 74 is able to control two functions: the distal-proximal position of spray head 42 and the channeling of irrigation fluid through

Button 58 is movable between a first configuration in which a flange 76 of button 58 engages an inner surface 78 of slider 74 and slider 46 is fixed relative to slider 74 and a second configuration in which flange 76 is spaced apart from inner surface 78 and slider 46 is movable relative to slider 74. This allows slider 46 to be fixed relative to slider 74 at any portion along groove 72, according to the preference of the operator, and provides an extra range of movement in addition to the movement of slider 74 in grooves 70, 70A, thereby increasing or decreasing the total length of guidewire 38 as measured from slider 74. In some embodiments, groove 72 includes a stepped interface 79 at end 75 and a stepped interface 80 at end 77. Interfaces 79, 80 are configured to engage flange 76 when button 58 is in the second configuration to fix slider 46 relative to slider 74 at end 75 or end 77.

Button 60 is movable between a first configuration in which a flange 81 of button 60 engages surface 78 and slider 46 is fixed relative to slider 74 and a second configuration in flange 81 is spaced apart from inner surface 78 and slider 46 is movable relative to slider 74. allows slider 46 to be fixed relative to slider 74 at any portion along groove 72A, according to preference of the operator, and provides an extra range of movement in addition to the movement of slider 74 in grooves 70, 70A, thereby increasing or decreasing the total length of guidewire 38 as measured from slider 74. In some embodiments, groove 72A includes a stepped interface 79A at end 75A and a stepped interface 80A at end 77A. Interfaces 79A, 80A are configured to flange 81 when button 60 is in the second configuration to fix slider 46 relative to slider 74 at 75A or end 77A. In some embodiments, slider 46 is movable relative to slider 74 when either button 58 or button 60 has the second configurations. In some embodiments, slider 46 is relative to slider 74 only when button 58 and button 60 both have the second configurations.

In operation and use, slider 74 is moved within grooves 70, 70A in the direction shown by arrow B or the direction shown by arrow C. Slider 46 is activated by pressing button 58 and/or button 60 inwardly such that flange 76 and/or flange 81 is/are spaced apart from surface 78. This allows buttons 58, 60 to move within grooves 72, 72A in the directions shown by arrows B and C. Once slider 46 is a selected position relative to grooves 72, 72A, buttons 58 and/or 60 are released such that flange 76 and/or flange 81 engage surface 78 to fix slider 46 relative to slider 74. This provides an extra range of movement in addition to the movement of slider 74 in grooves 70, 70A, thereby increasing or decreasing the total length of guidewire 38 as measured from slider 74. In some embodiments, movement of slider 46 within slider 74 provides for movement of around 10-15 mm.

In some embodiments, slider 46 includes an opening 82 extending into a sidewall 83 that is in communication with recess 52 and an opening 84 extending into sidewall 83 that is in communication with recess 54. Openings 82, 84 each extend parallel to axis A and are spaced apart from one another by wall 56. Button 58 includes an outward projection 86 configured for disposal in opening 82 to prevent button 58 from being removed from recess 52 as button 58 moves in the directions shown by arrows D and E. Button 60 includes an outward projection 88 configured for disposal in opening 84 to prevent button 60 from being removed from recess 54 as the button moves in the directions shown by arrows D and E. In some embodiments, buttons 58, 60 comprise a deformable and/or resilient material which allows projections 86, 88 to each be moved inwardly toward longitudinal axes defined by buttons 58, 60, respectively. Buttons 58, 60 are positioned in recesses 52, 54, respectively, when in the deformed configuration. Button 58 is moved relative to slider 46 in the direction shown by arrow D until projection 86 is aligned opening 82, at which time projection 86 snaps into opening 82 to prevent button 58 from being removed from recess 52. Button 60 is moved relative to slider 46 in the direction shown by E until projection 88 is aligned opening 84, at which time projection 88 snaps into opening 84 to prevent button 60 from being removed from recess 54. In some embodiments, opening 82 and/or opening 84 may be disposed at alternate orientations, relative to axis A, such as, for example, transverse, perpendicular and/or other angular orientations such as acute or obtuse, co-axial it may be offset or staggered. In some embodiments, button 58 and/or button 60 can be variously connected with slider 46, such as, for example, monolithic, integral connection, frictional engagement, threaded engagement, mutual grooves, screws, adhesive, nails, barbs and/or raised element.

To control device 10 and switch between its various modes of operation, handle 12 acts to switch device between an irrigation mode, an aspiration mode and a working channel clearing mode. In the irrigation mode, spray head 42 is located at the distal edge of passageway 36 creating a virtual nozzle spray. Irrigation fluid is caused to flow through passageway 36. Control of the flow may be accomplished either semi-automatically by pressing a button with predefined flow and pressure levels, or by pressing a pedal switch. In the aspiration mode, spray head 42 is located outside of passageway 36, preferably 5-20 mm on the distal side thereof. Vacuum pressure is activated and liquid and feces remains are aspirated through passageway 36 while device 10 is still inside the body cavity that is being cleansed. In the working channel clearing mode, spray head 46 is positioned such that it completely seals the distal exit of passageway 36. In addition to the vacuum pressure, a distally-directed positive flow pressure is activated through passageway 36, in order to assist in the aspiration process and prevent or clear blockage of passageway 36 by debris. The three operating modes are capable of serving two key functions: a) movement of the distal spray head between three different locations; and b) diversion of the irrigation fluid into the desired route (i.e. into the lumen of the partial-length tube during working channel clearing and directly into the working channel during irrigation).

In some embodiments, manifold 17 includes a water box, which contains the elements required for directing the irrigation fluid flow from a pump to the distal end of device 10 along the relevant pathways. In particular, the mechanism housed in the water box enables routing of the irrigation fluid between irrigation and water channel clearing (power irrigation) modes. The water box includes a water valve located close to an irrigation fluid inlet, which receives irrigation fluid pumped from a fluid reservoir or other externally-located fluid source. This fluid input will then be directed (in accordance with the position of a water valve lever) to one or both of two fluid channel routes: the space between tube 30 and tube 22 and/or passageway 36.

The tubing and water routing may be configured in two main modes: 1) irrigation the inner tube lumen always open; 2) irrigation through the inner tube lumen either open or The water valve lever is positioned such that the irrigation fluid is caused to flow simultaneously through two channels: passageway 36 (channel A) and the space located between tubes 22, 30 (channel B). This irrigation mode is achieved by means of the operator pushing 74 forward (i.e. distally) and then retracting slightly, in order that spray head 42 comes to rest on the distal outlet passageway 36. Simultaneously, guidewire 38 will move the water valve into the desired whereby both channels A and B are brought into fluid communication with the irrigation fluid source. In this state, the irrigation fluid that is pumped distally through channels A and B will through the apertures in spray head 42 in the form of a fluid spray which may be used to cleanse region of the body cavity (e.g. colon) that lies immediately beyond the distal end of the working channel.

Device 10 may then be switched from the irrigation mode to the power aspiration mode by means of the operator causing further retraction (i.e. proximal movement) of slider 74, such that spray head 42 is more tightly compressed against the exit of passageway 36, thereby closing apertures on spray head 42, and thus preventing any further fluid transfer across spray head 42. The corresponding movement of guidewire causes a valve to be rotated to a position in which channel B is closed, while channel A remains in the open position. In this mode, the pumped fluid passes through the lumen of tube 30 (i.e. channel A). Negative pressure is then applied to an aspiration inlet in handle 12 that is (in this mode) in fluid contact with channel B. In this way, irrigation fluid that exits the distal end of passageway 36 is aspirated, proximally, through the space between tubes 22, 30 (channel B).

In the second of the main modes mentioned hereinabove, passageway 36 may be in either closed or open states (in relation to the irrigation fluid inlet) in accordance with the position of the water valve lever. Channel A is open, thereby allowing irrigation fluid to pass distally through passageway 36. In some embodiments, however, channel A is closed and channel B is open, in which state the irrigation fluid inlet is in fluid contact only with the space between tubes 22, 30. In some embodiments, handle 12 may be used in conjunction with several different elongate instruments that are designed for insertion into a body passage (such as the colon). Thus, in the above description of the fluid channeling within manifold 17 (water box) of handle 12, the tube 22 is to be understood to also include within its scope other tubular structures including (but not limited to) the internal wall of a body passage (such as the colon or other part of the gastrointestinal tract), and other non-natural tubular elements such as the internal wall of an endoscopic channel (e.g., the working channel of a colonoscope).

As discussed above, slider 74 may be manipulated by the operator in order to change the operating mode of device 10. When device 10 is in aspiration mode, slider 74 is in its forward-most position, and negative aspiration pressure is applied to the space between tubes 22, 30. Movement of slider 74 into a more proximal location brings device 10 into irrigation mode, in which irrigation fluid is pumped distally through the space between tubes 22, 30 (and in the ‘always open’ mode, also through passageway 36). Finally, further proximal movement of slider 74 brings device 10 into its power aspiration mode, in which irrigation fluid is pumped distally through passageway 36 against a completely sealed spray head 42, and then aspirated proximally through the space between tubes 22, 30.

The present disclosure includes a method for cleansing a body passage comprising: a) inserting device 10 into said body passage such that the distal end thereof becomes located close to, and on the proximal side of, the area of said passage to be cleansed; b) passing spray head 42 through passageway 36 such that spray head 42 becomes located beyond the distal exit of passageway 36; c) introducing irrigation fluid into passageway 36 at a pressure that is sufficient to cause said fluid to form a spray or jet upon passing through channels, apertures and/or nozzles formed in spray head 42; d) allowing said spray or jet to cleanse the region of the body passage that is situated immediately distal to the end of passageway 36; e) causing spray head 42 to move distally such that there is no contact between spray head 42 and a distal face of passageway 36; f) applying a negative pressure to a proximal end of passageway 36, in order to cause aspiration of fluid and solid particulate matter through passageway 36; g) if necessary, bringing spray head 42 back to the location defined in step (b) and repeating steps (c) through (f).

In some embodiments, the step of passing spray head 42 through passageway 36 such that spray head 42 becomes located beyond the distal exit of passageway 36 includes moving 74 within grooves 70, 70A in the direction shown by arrow B or the direction shown by arrow C provide a first range of motion of guidewire 38 and/or spray head 42 relative to handle 12. In embodiments, the step of passing spray head 42 through passageway 36 such that spray head 42 becomes located beyond the distal exit of passageway 36 includes activating slider 46 by button 58 and/or button 60 inwardly such that flange 76 and/or flange 81 is/are spaced apart from surface 78. This allows buttons 58, 60 to move within grooves 72, 72A in the directions shown arrows B and C. Once slider 46 is a selected position relative to grooves 72, 72A, buttons 58 and/or 60 are released such that flange 76 and/or flange 81 engage surface 78 to fix slider 46 relative to slider 74. This provides second range of movement of guidewire 38 and/or spray head 42 relative to handle 12 in addition to the movement of slider 74 in grooves 70, 70A, thereby increasing or decreasing the total length of guidewire 38 as measured from slider 74. In some embodiments, movement of slider 46 within slider 74 provides for movement of around 10-15 mm.

In some embodiments, the method for cleansing a body passage includes i) withdrawing spray head 42 into passageway 36 such that spray head 42 is radially compressed, thereby causing its external diameter to be reduced, thereby sealing a distal end of passageway 36; and ii) introducing irrigation fluid into passageway 36 such that when the fluid leaves the distal end of tube 30, the positive fluid pressure provided thereby assists in preventing or removing blockages in the distal portion of the internal channel of the elongate medical device.

In some embodiments, the fluid spray jets produced by the spray head 42 nozzles or channels may be employed to facilitate the insertion of an endoscope into a body passage. Thus, the spray jets may assist by moving the GI tract folds and straightening the folds, thereby enabling easier pushing and pulling of the endoscope. The spray jets may also assist in endoscope insertion in some instances by means of their use to clear the GI tract lumen of large polyps, stones or other obstructions that may be located distally of the advancing distal tip of the endoscope. In addition to moving said obstructions, in some circumstances (e.g. in the case of certain stones) the fluid spray jets will also be able to cause their disintegration.

In embodiments where spray head 42 includes an O-ring, as discussed above, the method for cleansing a body passage includes pulling the spray head 42 into passageway 36 to a greater degree than described above (i.e. in relation to the irrigation stage), such that the O-ring is pulled into passageway 36, thereby totally blocking the exit of passageway 36. Once the exit of passageway 36 is blocked and passageway 36 is partially filled with feces and other debris, a high positive pressure pulse of liquid (or air) may be directed distally through passageway 36, thereby applying positive pressure to the distal part of passageway 36, and thus applying much higher forces to the solid debris than would otherwise be possible (i.e. by the use of a maximum negative 1 atmosphere produced by the vacuum force).

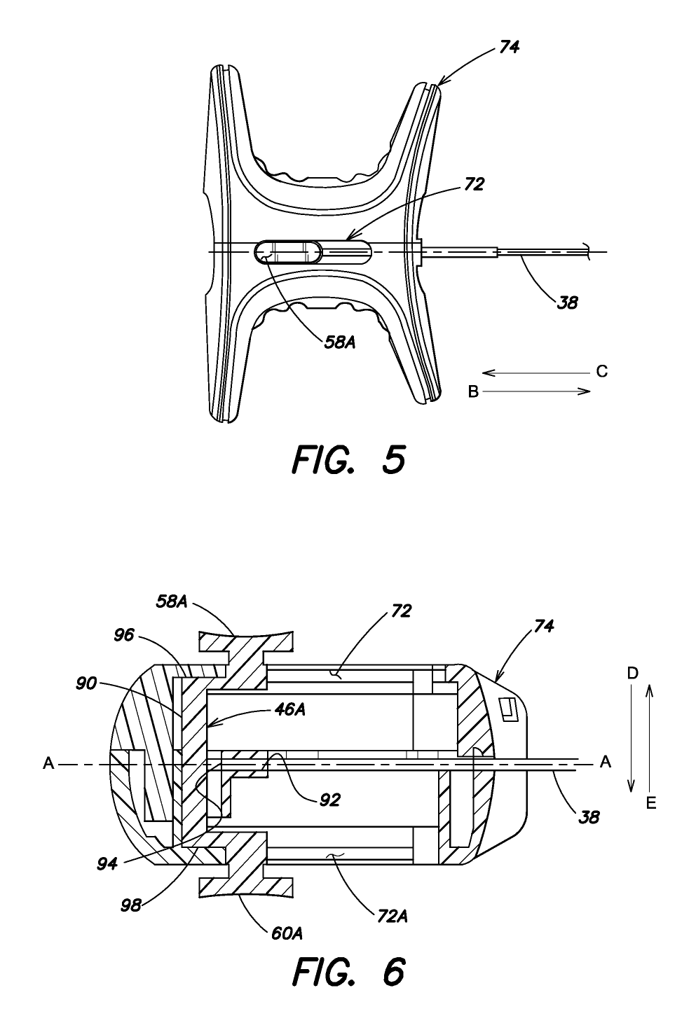

In one embodiment, shown in FIGS. 5 and 6, a slider 46A similar to slider 46 is disposed in slider 74. Slider 46A includes a body 90 including an inner surface 92 defining an aperture 94 that extends parallel to axis A. Guidewire 38 is disposed in aperture 94. In some embodiments, aperture 94 may be disposed at alternate orientations, relative to axis A, such as, example, transverse, perpendicular and/or other angular orientations such as acute or obtuse, co-co-axial and/or may be offset or staggered. In some embodiments, guidewire 38 can be variously connected with slider 46A, such as, for example, monolithic, integral connection, frictional engagement, threaded engagement, mutual grooves, screws, adhesive, nails, barbs and/or raised element.

An end 96 of body 90 includes a button 58A similar to button 58 extending therefrom and an end 98 of body opposite end 96 includes a button 60A similar to button 60 extending therefrom. Button 58A is integrally formed with body 90 such that button 58A and body 90 are monolithic. Button 60A is integrally formed with body 90 such that button 60A and body 90 are monolithic. That is, body 90 and buttons 58A, 60A are a single flexible element. Body 90 and buttons 58A, 60A are formed from a flexible material such that buttons 58A, 60A are each deformable relative to body 90. In some embodiments, body 90 and buttons 58A, 60A are formed from a flexible polymeric material.

In operation and use, slider 74 is moved within grooves 70, 70A in the direction shown by arrow B or the direction shown by arrow C. Slider 46 is activated by pressing button 58A in the direction shown by arrow E and pressing button 60A in the direction shown by arrow D such that buttons 58A, 60A move toward one another. This allows buttons slider 46A to move within grooves 72, 72A in the directions shown by arrows B and C. Once slider 46A is a selected position relative to grooves 72, 72A, buttons 58A and/or 60A are released to fix slider 46A relative to slider 74. This provides an extra range of movement in addition to the movement of slider 74 in grooves 70, 70A thereby increasing or decreasing the total length of guidewire 38 as measured from slider 74.

In order to operate device 10, additional items of equipment (such as a pump and a tank) are required. In one embodiment, these additional items of equipment may be conveniently contained within a discrete housing. This housing is connected by suitable tubing to a water tank used for supplying the irrigation fluid to device 10. In some embodiments, device 10 may withstand irrigation pressures of up to 15 atmospheres and flow rates of about 3 liter/min. The streamed washing fluid may be controlled by the operator by means of pedal switch electrically connected to the console. The console is also connected to the working channel of a colonoscope for applying vacuum therethrough by means of a vacuum pump and aspirating debris, fecal material and other particulate matter into a waste tank.

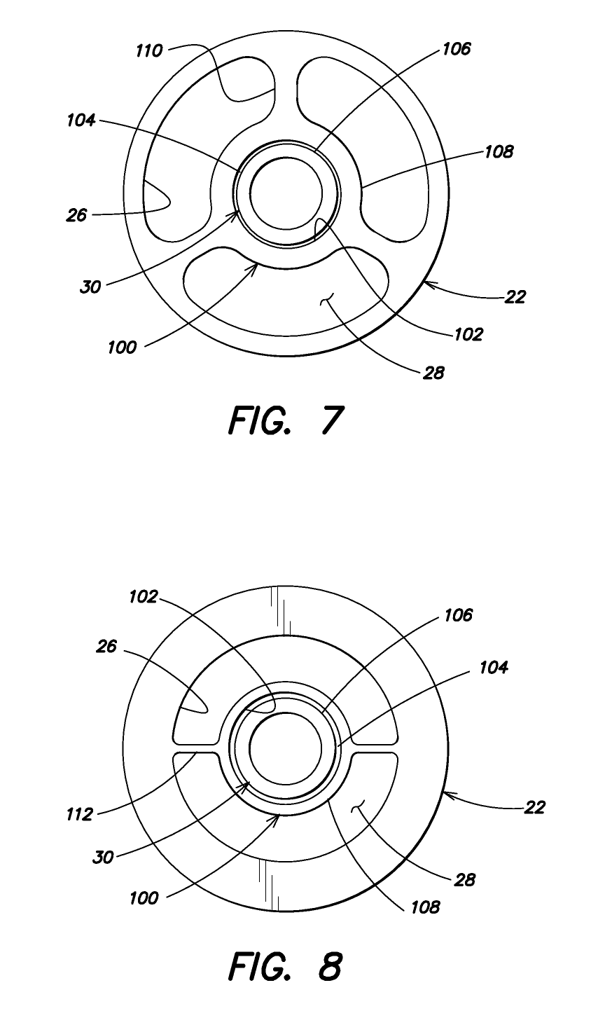

In one embodiment, shown in FIG. 7, tube 22 incorporates structures that maintain tube 30 within passageway 28 such that tube 30 is maintained in a coaxial configuration with tube 22 and/or lateral movement of tube 30 relative to tube 22 is limited. In particular, tube 22 includes an inner member 100 that is coaxial with tube 22. Member 100 has an inner surface 102 defining a centralizing lumen or channel 104 having a cylindrical configuration that is coaxial with axis A and tube 22. Channel 104 is configured for disposal of tube 30. In some embodiments, surface 102 engages an outer surface 106 of tube 30 to prevent lateral movement of tube 30 within channel 104. In some embodiments, channel 104 has a diameter that is greater than that of tube 30 to allow tube 30 at least some degree of lateral movement within channel 30. An outer surface 108 of member 100 includes a plurality of spaced apart ribs 110 extending therefrom. Ribs 110 each extend from surface 108 to surface 26 to maintain the position of member 100 relative to surface 26. Ribs 110 are disposed radially about member 100 and are spaced apart from one another. In some embodiments, device 10 may include on or a plurality of ribs 110. In some embodiments, member 100 and/or ribs 110 extend along the entire length of tube 22. In some embodiments, member 100 and/or ribs 110 extend along less than the entire length of tube 22. In some embodiments, tube 22 includes a first portion including member 100 and ribs 110 and a second portion including member 100 and ribs 110, the first portion being spaced apart from the second portion by a third portion that does not include member 100 and ribs 110. In some embodiments, an end 118 of tube 22 and/or an end 120 of tube 22 opposite end 118 define a portion 122 of tube 22 that does not include member 100 or ribs 110 to facilitate insertion of a component into passageway 28 and/or to facilitate engagement of tube 22 with handle 12. In some embodiments, ribs 110 are equidistantly spaced from one another. In some embodiments, member 100 and/or channel 104 may be disposed at alternate orientations, relative to axis A and/or tube 22, such as, for example, transverse, perpendicular and/or other angular orientations such as acute or obtuse, co-axial and/or may be offset or staggered. In some embodiments, tube 30 can be variously connected with member 100, such as, for example, monolithic, integral connection, frictional engagement, threaded engagement, mutual grooves, screws, adhesive, nails, barbs and/or raised element.

In one embodiment, shown in FIG. 8, tube 22 incorporates structures that maintain tube 30 within passageway 28 such that tube 30 is maintained in a coaxial configuration with tube 22 and/or lateral movement of tube 30 relative to tube 22 is limited. In particular, surface 108 includes a pair of spaced apart ribs 112 extending therefrom. Ribs 112 each extend from surface 108 to surface 26 to maintain the position of member 100 relative to surface 26. Ribs 112 are equidistantly spaced from one another such that ribs 112 extend parallel to one another. In some embodiments, member 100 and/or ribs 112 extend along the entire length of tube 22. In some embodiments, member 100 and/or ribs 112 extend along less than the entire length of tube 22. In some embodiments, tube 22 includes a first portion including member 100 and ribs 112 and a second portion including member 100 and ribs 112, the first portion being spaced apart from the second portion by a third portion that does not include ribs 112. In some embodiments, an end 118 of tube 22 and/or an end 120 of tube 22 opposite end 118 define a portion 122 of tube 22 that does not include member 100 or ribs 112 to facilitate insertion of a component into passageway 28 and/or to facilitate engagement of tube 22 with handle 12.

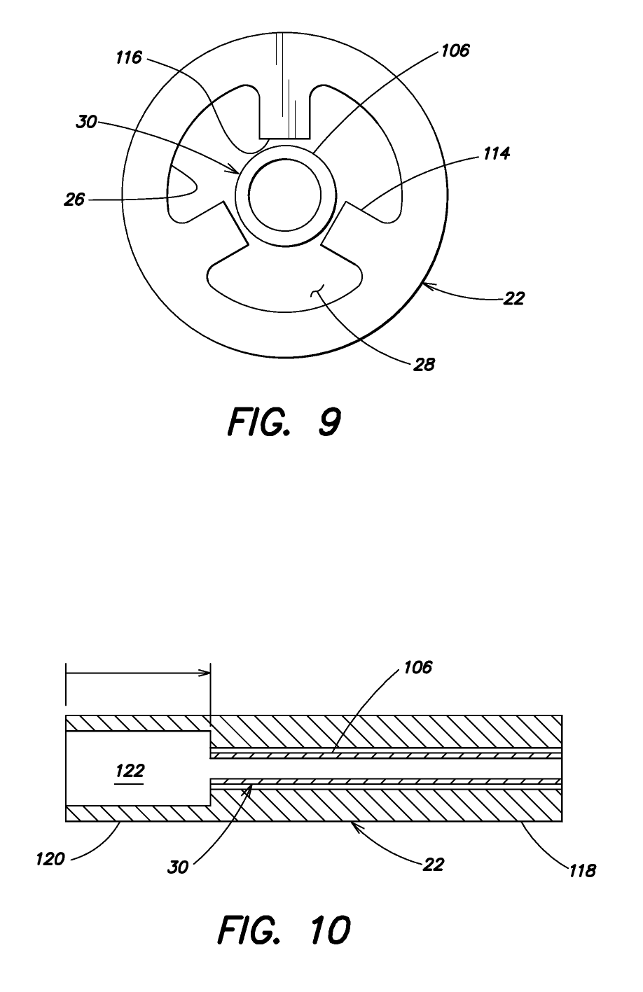

In one embodiment, shown in FIGS. 9 and 10, tube 22 incorporates structures that maintain tube 30 within passageway 28 such that tube 30 is maintained in a coaxial configuration with tube 22 and/or lateral movement of tube 30 relative to tube 22 is limited. In particular, 26 includes a plurality of spaced apart ribs 114 extending inwardly therefrom such that ribs 114 extend toward axis A. Ribs 114 each extend from surface 26 and are spaced apart from tube 30. Ribs 114 each include a planar end surface 116 configured to engage surface 106 maintain tube within passageway 28 such that tube 30 is maintained in a coaxial configuration with tube 22 and/or lateral movement of tube 30 relative to tube 22 is limited. Ribs 114 are disposed radially about tube 22 and are spaced apart from one another. In some embodiments, the device may include on or a plurality of ribs 114. In some embodiments, ribs 114 extend along the entire of tube 22. In some embodiments, ribs 114 extend along less than the entire length of tube 22. some embodiments, tube 22 includes a first portion including ribs 114 and a second portion including ribs 110, the first portion being spaced apart from the second portion by a third portion that does not include ribs 114. In some embodiments, an end 118 of tube 22 and/or an end 120 tube 22 opposite end 118 define a portion 122 of tube 22 that does not include ribs 114 to insertion of a component into passageway 28 and/or to facilitate engagement of tube 22 with handle 12. In some embodiments, ribs 114 are equidistantly spaced from one another. In some embodiments, all or only a portion of surfaces 116 may be variously configured and such as, for example, planar, concave, polygonal, irregular, uniform, non-uniform, staggered, tapered, consistent or variable.

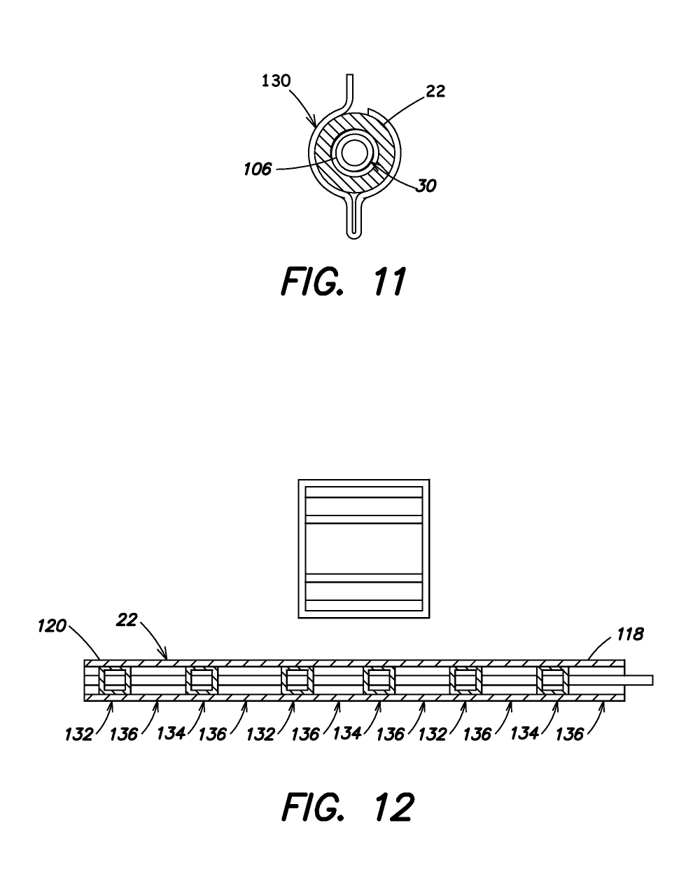

In one embodiment, shown in FIGS. 11 and 12, tube 22 incorporates structures that maintain tube 30 within passageway 28 such that tube 30 is maintained in a coaxial configuration with tube 22 and/or lateral movement of tube 30 relative to tube 22 is limited. In particular, the device includes a spring, such as, for example, a leaf spring 130 positioned between tube 22 and tube 30. In some embodiments, spring 130 can be variously connected with surface 26, such as, for example, monolithic, integral connection, frictional engagement, threaded engagement, mutual grooves, screws, adhesive, nails, barbs and/or raised element. Spring 130 extends radially about surface 26 such that an inner surface of spring 130 engages surface 106. In some embodiments, the inner surface of spring 130 is slightly spaced apart from surface 106 so that tube 30 can translate laterally, at least to some degree, within passageway 28. In some embodiments, spring 130 extends along the entire length of tube 22. In some embodiments, spring 130 extends along less than the entire length of tube 22. In some embodiments, spring 130 includes a first portion 132 and a second portion 134, first portion 132 being spaced apart from second portion 134 by a third portion 136 of tube 22 that does not include spring 130. In some embodiments, device 10 includes a plurality of first portions 132, second portions 134 and third portions 136, as shown in FIG. 12. In some embodiments, end 118 and/or end 120 define a third portion 136 that does not include spring 130 to facilitate insertion of a component into passageway 28 and/or to facilitate engagement of tube 22 with handle 12.

In some embodiments, the spring can stabilize the inner tube, outer tube and/or guidewire and thus prevent lateral and/or vertical movement of the guidewire, spray head and/or plug, particularly when one or more of these components are in or outside of the working channel of the endoscope.

It will be understood by those of ordinary skill in the art that the body passage device and one or more of its components can be sterilized and reused. Alternatively, and more preferred, one or more components of the body passage device can be disposable and disposed of after single use.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. A body passage device, comprising:
a housing configured to receive a guidewire, the housing having a longitudinal axis;
a first guidewire adjuster contacting the housing and having a proximal end and a distal end, the first guidewire adjuster configured to extend the guidewire a first length along the longitudinal axis of the housing; and
a second guidewire adjuster configured to contact the proximal end and/or the distal end of the first guidewire adjuster and/or guidewire and extend the guidewire a second length along the longitudinal axis of the housing.

2. A body passage device according to claim 1, wherein (i) the second guidewire adjuster is disposed within the first guidewire adjuster and the first guidewire adjuster is slidably mounted along the longitudinal axis of the housing to extend the guidewire the first length; or (ii) the second guidewire adjuster is slidably mounted along the longitudinal axis of the housing to extend the guidewire the second length.

3. A body passage device according to claim 1, wherein (i) the first guidewire adjuster is configured to engage the guidewire to extend the guidewire the first length; (ii) the first guidewire adjuster is configured to engage the second guidewire adjuster to extend the guidewire the first length; or (iii) the first guidewire adjuster is configured to engage the guidewire and the second guidewire adjuster to extend the guidewire the first length.

4. A body passage device according to claim 1, wherein the second guidewire adjuster comprises a lock to hold the guidewire in a discrete position.

5. A body passage device according to claim 4, wherein the lock comprises a spring, button or knob to hold the guidewire in the discrete position.

6. A body passage device according to claim 1, wherein (i) the first guidewire adjuster is parallel to the second guidewire adjuster; (ii) the guidewire comprises a spray head at its distal end; or (iii) the guidewire is configured to be inserted into a working channel of an endoscope.

7. A body passage device, comprising:
an outer tube having proximal and distal ends and a longitudinal axis disposed therebetween, the outer tube having a channel therein extending along the longitudinal axis of the outer tube;
a guidewire disposed in the channel and configured to extend through the proximal end of the outer tube and through and beyond the distal end of the outer tube; and
a stabilizing member configured to prevent lateral and/or vertical movement of the guidewire relative to the channel of the outer tube.

8. A body passage device of claim 7, wherein (i) the guidewire is configured to be inserted into a working channel of an endoscope; or (ii) the guidewire comprises a spray head at its distal end.

9. A body passage device of claim 7, wherein the stabilizing member comprises an inner tube having a portion that extends coaxially in the channel of the outer tube to reduce lateral and/or vertical movement of the guidewire.

10. A body passage device of claim 7, wherein the stabilizing member comprises (i) a ring disposed in the channel of the outer tube and configured to contact the guidewire to reduce lateral and/or vertical movement of the guidewire; or (ii) a ring disposed in the channel of the outer tube having a portion that extends coaxially in the channel of the outer tube to reduce lateral and/or vertical movement of the guidewire; or (iii) a ring disposed in the channel of the outer tube and configured to contact an inner tube to reduce lateral and/or vertical movement of the guidewire.

11. A body passage device of claim 7, wherein the stabilizing member comprises (i) one or more ribs or projections disposed in the channel of the outer tube to reduce lateral and/or vertical movement of the guidewire; or (ii) one or more ribs or projections disposed in the channel of the outer tube having a portion that extends coaxially in the channel of the outer tube to reduce lateral and/or vertical movement of the guidewire; or (iii) one or more ribs or projections disposed in the channel of the outer tube and configured to contact an inner tube to reduce lateral and/or vertical movement of the guidewire.

12. A body passage device of claim 7, wherein the stabilizing member comprises (i) one or more springs disposed in the channel of the outer tube to reduce lateral and/or vertical movement of the guidewire; or (ii) one or more springs disposed in the channel of the outer tube having a portion that extends coaxially in the channel of the outer tube to reduce lateral and/or vertical movement of the guidewire; or (iii) one or more springs disposed in the channel of the outer tube and configured to contact an inner tube to reduce lateral and/or vertical movement of the guidewire.

13. A method of performing an endoscopy, the method comprising inserting the guidewire of the body passage device of claim 1 into a working channel of an endoscope to clean or irrigate the working channel or a patient tissue.

14. A method of performing an endoscopy of claim 13, wherein the patient tissue is a colon or esophagus.

15. A body passage cleaning device, comprising:
a tube comprising an inner surface defining a channel;
an actuating element movably disposed in the channel, the actuating element extending along a longitudinal axis between a first end and a second end, the second end including a plug; and
a handle coupled to the tube, the handle comprising an inner surface defining a slot, the slot having an internal slider movably disposed therein such that the first end engages the internal slider, wherein the internal slider comprises an inner surface defining a hole extending perpendicular to the longitudinal axis, at least a portion of the first end being disposed in the hole.

16. A device according to claim 15, wherein the internal slider is configured to adjust a distance between the handle and the plug.

17. A device according to claim 15, wherein a first button is coupled to a first side of the slider and a second button is coupled to a second side of the slider; the first button extends through a groove in a first side of the handle and the second button extends through a groove in a second side of the handle; and the buttons are movable between a first configuration in which flanges of each of the buttons engage an inner surface of an external slider that is coupled to the internal slider and the internal slider is fixed relative to the external slider and a second configuration in which the flanges are spaced apart from the inner surface of the external slider and the internal slider is movable relative to the external slider.

18. A device according to claim 17, wherein the buttons are each spring-loaded.

19. A device according to claim 17, wherein the slider comprises a pair of spaced apart recesses each extending parallel to the longitudinal axis; and each of the buttons comprises a projection configured for disposal in one of the recesses to prevent the buttons from falling out of the grooves when the buttons are in the first configuration.

20. A device according to claim 17, wherein the first button is integrally formed with the first side of the internal slider and the second button is integrally formed with the second side of the internal slider.

21. A device according to claim 20, wherein the first button is deformable relative to the second button to move the buttons between the first and second configurations.

22. A device according to claim 20, wherein the buttons and the slider are each formed from a common flexible material such that the buttons and the slider define a single flexible element.

23. A device according to claim 22, wherein the flexible material is a flexible polymeric material.

24. A body passage cleaning device, comprising:
an outer tube comprising an inner surface defining a first channel;
an inner tube disposed in the first channel, the inner tube comprising an inner surface defining a second channel; and
an actuating element movably disposed in the second channel, the actuating element comprising a first end and a second end including a plug,
wherein the inner tube is positioned relative to the outer tube such that the inner tube is coaxial with the outer tube.

25. A device according to claim 24, wherein the device comprises a plurality of ribs connecting the inner surface of the outer tube with an outer surface of the inner tube to fix the inner tube relative to the outer tube such that the inner tube is coaxial with the outer tube.

26. A device according to claim 25, wherein the plurality of ribs comprises three ribs that are spaced equidistant from one another.

27. A device according to claim 25, wherein the plurality of ribs comprises two ribs that are spaced equidistant from one another.

28. A device according to claim 25, wherein the ribs are integrally formed with the inner and outer tubes.

29. A device according to claim 25, wherein the device comprises a plurality of ribs extending from the inner surface of the outer tube such that the ribs are spaced apart from an outer surface of the inner tube; and lateral movement of the inner tube relative to the outer tube causes at least one of the ribs to engage the outer surface of the inner tube to position the inner tube relative to the outer tube such that the inner tube is at least substantially coaxial with the outer tube.

30. A device according to claim 29, wherein the outer tube has a length defined by a distance between first and second ends of the outer tube; and the ribs extend along a portion of the length of the outer tube that is less than the entire length of the outer tube.

31. A device according to claim 25, wherein the device comprises a series of leaf springs assembled on an outer surface of the inner tube that engage the inner surface of the outer tube to position the inner tube relative to the outer tube such that the inner tube is at least substantially coaxial with the outer tube.

32. A body passage cleaning device, comprising: an outer tube comprising an inner surface defining a first channel; an inner tube disposed in the first channel, the inner tube comprising an inner surface defining a second channel, wherein a plurality of ribs extending from the inner surface of the outer tube engage an outer surface of the inner tube to position the inner tube relative to the outer tube such that the inner tube is coaxial with the outer tube; an actuating element movably disposed in the second channel, the actuating element comprising a first end and a second end including a plug; and a handle coupled to the inner tube, the handle comprising an inner surface defining a slot, the slot having an internal slider movably disposed therein such that the first end engages the internal slider, wherein the slider comprises an inner surface defining a hole extending perpendicular to the longitudinal axis, at least a portion of the first end being disposed in the hole.

33. A device according to claim 32, wherein a first button is coupled to a first side of the internal slider and a second button is coupled to a second side of the internal slider; the first button extends through a groove in a first side of the handle and the second button extends through a groove in a second side of the handle; and the buttons are movable between a first configuration in which flanges of each of the buttons engage an inner surface of an external slider that is coupled to the internal slider and the internal slider is fixed relative to the external slider and a second configuration in which the flanges are spaced apart from the inner surface of the external slider and the internal slider is movable relative to the external slider.

34. A device according to claim 32, wherein the outer tube has a length defined by a distance between first and second ends of the outer tube; and the ribs extend along a portion of the length of the outer tube that is less than the entire length of the outer tube.

35. (canceled)
